(19) United States
(12) Patent Application Publication
(10) Pub. No.: US 2011/0066238 A1
(43) Pub. Date: Mar. 17, 2011

(54) RECONSTRUCTED CORNEA AND MUCOUS MEMBRANE

(75) Inventors: Nicolas Bechetoille, Brignais (FR); Odile Damour, Saint Genis Laval (FR); Valerie Andre, Ludwigshafen (DE)

(73) Assignee: BASF BEUTY CARE SOLUTIONS FRANCE S.A.S, LYON (FR)

(21) Appl. No.: 12/867,384

(22) PCT Filed: Feb. 16, 2009
(86) PCT No.: PCT/EP09/51823

§ 371 Date: Nov. 30, 2010

(30) Foreign Application Priority Data
Feb. 14, 2008 (FR) 0850948

(51) Int. Cl. A61F 2/14; C12N 5/071; C12M 1/18

(52) U.S. Cl. 623/5.11; 435/396; 435/289.1

(57) ABSTRACT

The invention relates to a model of reconstructed cornea, which may be used especially in tissue engineering, to a biomaterial that may be used for preparing a reconstructed cornea, and also to a culture device allowing better reproducibility of the cultures of the model of reconstructed cornea.

The invention relates to a model of reconstructed cornea in tissue engineering, to a biomaterial that may be used for the preparation of reconstructed cornea, and also to a culture device allowing better reproducibility of cultures of the model of reconstructed cornea as well as reconstructed corneal stroma.

The invention also relates to a model of mucous membrane and to a culture device for preparing this model.

### PRIOR ART

The reconstruction of corneas by tissue engineering has the aim of obtaining pharmacotoxicological models and of progressing towards their therapeutic use (Germain et al. 2000; Carlsson et al. 2003). The current models are constituted of a support, generally a collagen gel, in which are dispersed keratocytes to reconstitute a stroma. This stroma is subsequently seeded with epithelial cells and occasionally endothelial cells to reconstitute an artificial cornea. It is essential to have cells in sufficient number and well characterized. Two methods exist for obtaining such cultures: using transfected lines, or starting the culturing with primary human cells. The latter method has the twofold advantage of giving models that are closer to physiology and of opening the way to possible therapies.

Only the corneal epithelial stem cells, located on the limbus, have been identified and characterized (Pellegrini et al. 1999; 2001). The culturing of endothelial cells is in its infancy and the amounts obtained remain modest due to their low proliferative capacity. Endothelial stem cells have not been identified, although the presence of endothelial progenitors close to the Schwalbe line is suspected. The endothelial cells used in the models are derived from transfected lines. (Reichl et al.; Griffith et al. 1999).

Keratocytes are quiescent in the physiological state and are morphologically well identified in situ. They have different phenotypes depending on the environmental conditions. There are at least three keratocyte phenotypes in vivo: the quiescent keratocyte, the active keratocyte (fibroblast) and the myofibroblast (Musselmann et al. 2003). To these three phenotypes are added three types of keratocyte that are morphologically different as a function of their location in the corneal thickness. In man, there are morphometric differences between the keratocytes of the anterior stroma (0-200 μm), median stroma (200-400 μm) and posterior stroma (400-600 μm). The mean size of the cells in situ, when they are flattened and spread out, is about 300-400 μm (measured by transmission electron microscopy). When these cells are in suspension, they become spherical and their size is much smaller but remains proportional to the size in situ (about 5-20 μm). The greatest difference between these three populations is the cell volume: the anterior and central keratocytes have an identical volume of about 5×10³ μm³, whereas the keratocytes of the posterior stroma have a larger volume, of about 14.4×10³ μm³ (Hahnel et al. 2000).

The phenotypes may be differentiated by certain markers: in particular, the antigen CD34 has been identified as a marker of the keratocyte phenotype in the cornea. Smooth muscle α-actin (α-SMA) is an actin isoform characteristic of the myofibroblast, which appears during the transformation of the keratocyte into a myofibroblast during the cicatrization process. The tendency towards apoptosis of myofibroblasts, which is normal in the cicatrization process, and its qualitative differences in collagens synthesis do not allow the reconstruction of a stroma in tissue engineering from this phenotype (Jester et al. 2003; Gabbiani 2003).

Other cell culture supports were previously described by the Applicant for culturing different cells types. But the biomaterial used in these supports has higher pore size with mean pores diameter around 150-200 microns. Such supports do not allow proper proliferation and thus do not permit good epithelialisation on the surface of the reconstructed stroma.

Thus the prior art does not propose a model that is really optimized for the culturing of corneal cells. At the present time, there is no satisfactory model of cornea and in particular no model of cornea with cellular recovery capability or cellular regeneration capability. The invention proposes to overcome these problems.

### AIMS OF THE INVENTION

The main aim of the invention is to overcome the new technical problem that consists in providing a novel support or device for culturing cells that are capable of forming a model of cornea, and/or a biomaterial of the reconstructed cornea type in tissue engineering, and/or a model of reconstructed corneal stroma and/or a model of reconstructed cornea.

The aim of the invention is to provide a model of reconstructed cornea having regeneration capability.

The aim of the invention is to provide such a culture device, in particular in an automated and industrial manner. Thus, the aim of the invention is to provide a sterile process for producing such a culture device, reproducibly, safely and reliably, on a large scale for industrial and medical use, especially in order to allow high-throughput screening to be performed.

The aim of the invention is also to provide a model of cornea, especially to be used as an alternative to toxicity tests on animals for cosmetological and pharmacological applications.

The aim of the invention is also to provide a model of mucous membrane, and in particular of cornea, which is competent for simulating a tissue repair, especially such as obtained on rabbit eye.

The aim of the invention is also to provide a reconstructed cornea especially for use in cornea grafts or corrective surgery.

### DESCRIPTION OF THE INVENTION

The invention allows cornea cells and/or mucous membrane cells to be cultured on or in a tissue support that is not a human cornea membrane, in an industrial, reliable, inexpensive and automatable manner.

In the context of the present invention and without specific mention, “cornea” has to be understood as a cornea at least partly reconstructed such as hemicornea and full reconstructed structure of cornea.

In the context of the present invention, “corneal stroma” has to be understood as a model of corneal stroma, especially a human cornea stroma, comprising at least corneal stromal cells, preferably corneal kerotocytes and a cell culture support for culturing corneal cells, said support comprising at least one biopolymer in the form of a porous matrix whose porosity is comprised between 10 and 100 microns.

According to a first aspect, the invention relates to a cell culture support (or cell culture substrate or biomaterial) for culturing corneal or mucous membrane cells, the said support comprising an aqueous gel, optionally dehydrated, of at least one biopolymer suitable for culturing corneal or mucous membrane stromal cells.

According to a preferred embodiment, the aqueous gel is dehydrated and has a porosity comprised between 10 and 100 microns, preferably between 30 and 70 microns and more preferably between 40 and 50 microns.

The aqueous gel, optionally dehydrated, is also suitable for culturing another cell type, for instance corneal or mucous membrane epithelial cells and/or endothelial cells.

Advantageously, the cells are human cells, for instance primary human cells or cells derived from cell lines.

Advantageously, the support according to the present invention comprises an aqueous gel based on an aqueous solution of at least one biopolymer.

The expression “material based on” means a material which comprises or is essentially constituted of or solely constituted of the material considered. Thus, for example, the material based on at least one biopolymer preferably comprises at least two biopolymers as a mixture.

The term “biopolymer” means a macromolecule that participates in structural and functional organization, in the metabolic process and in the maintenance of a live organism, and which is possibly synthesized by a living organism. A biopolymer generally comprises covalent bonds between amino acids, and/or nucleotides, and/or carbohydrates. The term “biopolymers” also means biopolymers that are identical to those found in nature, but are obtained by other means, for instance by synthesis, etc. A macromolecule that plays a role in the formation of the macromolecular structures of a living organism, and preferably in the extracellular matrix (ECM), is preferred. Thus, the preferred biopolymers are chosen from glycoproteins, such as collagen, fibronectin or laminin, or pure proteins, such as elastin, or polysaccharides, which are optionally substituted for instance chitin or chitosan. These biopolymers may of course be recombinant or isolated biopolymers (of artificial or natural origin).

Preferably, the aqueous gel is based on an aqueous solution of a macromolecule of the ECM, preferably at least one glycoprotein and more preferably based on an aqueous solution of collagen. Advantageously, the glycoprotein, and preferably a collagen, is combined with at least one polysaccharide, which is optionally substituted. The collagen is especially of bovine, porcine, equine or marine origin, and preferably a type I/III collagen optionally in combination with a type V collagen, which may or may not be supplemented with collagens IV and/or VI and/or VII.

The biopolymer, and preferably the glycoprotein, and more preferably the collagen, is preferably combined with one or more polysaccharides, which are optionally substituted, for instance a glycosaminoglycan, such as chondroitin 4-sulfate, chondroitin 6-sulfate or hyaluronic acid, or a mixture thereof, and/or chitin, and/or chitosan; and/or one or more proteoglycans. Preferably, at least one polysaccharide and chitosan, optionally modified, are combined.

The weight percentage distribution relative to the final dry matter between the biopolymers is:

collagen (60-90), chitosan (10-30), glycosaminoglycan (0-15); advantageously, collagen (62-72), chitosan (20-25), glycosaminoglycan (8-12) and in particular collagen (72), chitosan (20), glycosaminoglycan (8).

The cell culture support may be a gel or a dehydrated gel, especially forming a matrix or a sponge. The dehydration may be obtained by thermal dehydration or by freeze-drying.

The term “porosity” as used in the present invention has to be understood as a mean pore diameter. Mean pore diameter may be measured with methods know in prior art. For instance, it may be measured with scanning electron microscopy analysis at magnifications ranging from 50× to 7000×. Software such as Lucia™, version 5.02, marketed by Nikon, France may be used for image analysis, in particular under automatic measure.

The term “homogeneous repartition” as used in the present invention has to be understood as almost the same pore repartition within biomaterial. Manufacturing process may be adjusted to create or not pore gradient within biomaterial. Such homogeneous repartition may be obtained notably with fast freeze-drying step.

As previously mentioned, it has been discovered, entirely surprisingly, that a cell culture support prepared by dehydration of a gel comprising at least one biopolymer with a porosity comprised between 10 and 100 microns, preferably between 30 and 70 microns and more preferably between 40 and 50 microns allows very good culturing of corneal stromal cells, and makes it possible to have epithelialisation and to prepare a reconstructed cornea and/or reconstructed mucous membrane of good quality. It is preferred in this context to use as biopolymer a macromolecule of the ECM, and preferably collagen.

Among the polysaccharides, which are optionally substituted, that may advantageously be used is a glycosaminoglycan (GAG), optionally combined with a chitin or a chitosan.

Advantageously, the culture support is an aqueous gel based on a mixture of collagen, of at least one polysaccharide and of chitosan, optionally modified and allows very good culturing of corneal and/or mucous membrane stromal cells, especially when it is dehydrated.

An example of a modified chitosan that may be mentioned is a chitosan with a degree of acylation, preferably of acetylation, controlled as a function of the envisaged use, different degrees of acetylation being well known to those skilled in the art and in particular described in the abovementioned European document EP 0 296 078, which is incorporated herein in its entirety by reference.

The invention thus also relates to a porous matrix formed by this culture support, and allowing the culturing of corneal or mucous membrane stromal cells. It is referred to as a sponge due to the porous structure. The diversity of phenotypes and of forms of keratocytes has not been taken into account, to the inventors' knowledge to date, in the reconstruction of the stromal part of the current models, with the exclusion of myofibroblasts. This taking into account appears to be important for better cells culturing. The culture models currently proposed on the market are not suitable for culturing corneal cells. The present invention proposes a culture model that is suitable for culturing corneal cells.

In the present invention, the expression “suitable for culturing cells” means a model that allows a satisfactory proliferation for the needs of manufacturing a cell culture support or model.

In particular, the invention covers a cell culture support for culturing corneal cells, the said support comprising at least one biopolymer in the form of a porous matrix whose pore size is defined to allow corneal stromal cells to diffuse and adhere within the support and allow proliferation of corneal stromal cells, in particular of corneal keratinocytes. A reconstructed stroma suitable for proliferation of other different type cells and preferably for epithelialisation is obtained and provides cornea having cellular recovery capability, in particular epithelium regeneration capability. The porosity is thus comprised between 10 and 100 microns, preferably between 30 and 70 microns and more preferably between 40 and 50 microns.

The corneal keratocytes proliferation was notably illustrated and demonstrated in example 4.

This regeneration capability or cellular recovery was notably illustrated and demonstrated in examples 8 and 9 after SDS treatment.

The size of the pores of the present invention does not require a gradient insofar as the cells naturally penetrate the matrix after being deposited on the surface, and then adhere to the fibres of the support. Thus, it is not sought to make the cells penetrate by means of large-sized pores and then to make them adhere by shrinking the pore size as a function of the depth of the material. The size of the pores is large enough to allow the cells, and especially the stromal cells, to diffuse or penetrate within the matrix, but small enough for the cells to adhere to the walls and not to pass through the matrix without adhering thereto. Advantageously, the size distribution of the pores is homogeneous. The biomaterial manufacturing process described in the present invention allows such an homogeneous pores size distribution.

According to a preferred embodiment, pore size distribution is such that at least 50% of pores diameters have targeted porosity.

Advantageously, the pores are formed by removing solvent from a solution (dehydration in the case of an aqueous solution) comprising the biopolymer or biopolymers of the present invention. The biopolymer may be alone or in a mixture as explained in the present invention.

Advantageously, the porous matrix is based on a mixture of collagen, of at least one polysaccharide, and of chitosan, optionally modified, and preferably with a proportion of collagen of between 60% and 90%, of GAG of between 0 and 15%, and of chitosan of between 10% and 30%, as a dry weight percentage relative to the total dry weight of this mixture.

The process for preparing the porous matrix comprises the variants described in the present invention.

The parameters of the dehydration process depend on the porosity to be obtained, which is adapted to the sizes of the corneal cells or mucous membrane cells to be cultured. The invention thus relates to a cell culture support for culturing corneal or mucous membrane cells, the said support comprising an aqueous gel, optionally dehydrated, of at least one biopolymer suitable for culturing corneal or mucous membrane stromal cells, the said aqueous gel, which is optionally dehydrated, having a porosity suitable for culturing stromal cells, especially corneal stromal cells or mucous membrane stromal cells. The culture support generally has a porosity of between 10 and 100 microns (mean diameter). A porosity of between 30 and 70 microns and more particularly between 40 and 50 microns, for instance between 42 and 49 microns, is preferably targeted. The calculation of porosity was performed by scanning electron microscopy studies at magnifications ranging from 50× to 7000×.

Among the collagen gels that may be used are collagens of I/III type optionally in combination with a collagen of V type, which may or may not be supplemented with collagens IV and/or VI and/or VII. Advantageously, the culture support is an aqueous gel, optionally dehydrated, based on a mixture of collagen, glycosaminoglycan and chitosan, optionally modified, and has a concentration that is suitable for culturing stromal cells and in particular for obtaining a sponge after dehydration. This concentration is generally between 1.25% and 1.6% of the mixture of collagen, glycosaminoglycan and chitosan, optionally modified, relative to the aqueous medium (generally water, preferably laboratory water or distilled water). This concentration has an influence on the porosity of the sponge obtained after dehydration. Thus, the porosity depends on the viscosity of the gel.

Advantageously, the preparation of the layer of porous material is performed by freeze-drying the aqueous gel, especially by freezing at a temperature of between −30° C. and −196° C., and preferably, for industrial reasons, at a temperature of between −40° C. and −80° C. This temperature has an influence on the porosity of the support and should be adapted to the desired porosity.

The invention also relates to a cell culture device for culturing corneal or mucous membrane cells, comprising a recess and at least one support as defined previously forming a layer for culturing the cells to be cultured, this layer being obtained by gelling an aqueous solution of at least one biopolymer directly poured into the recess of the device.

According to one particular embodiment, the cell culture device for culturing corneal or mucous membrane cells comprises a recess and at least one support as defined previously forming a layer for culturing the cells to be cultured, this layer being obtained by dehydrating an aqueous gel of at least one biopolymer directly poured into the recess of the device.

The device described in the patent FR 2 881 434 is particularly suitable for the present invention, all the more so since it is suitable for an automated preparation of cell culture supports. This cell culture support device is suitable for industrial use and especially for high-throughput screening.

According to one embodiment, the device comprises a first zone for culturing stromal cells, mentioned as the “stromal zone”, and a second zone for culturing either epithelial cells, mentioned as the “epithelial zone”, or endothelial cells, mentioned as the “endothelial zone”. According to another embodiment, the device comprises a first zone for culturing stromal cells, mentioned as the “stromal zone”, and a second zone for culturing epithelial cells, mentioned as the “epithelial zone”, and a third zone for culturing endothelial cells, mentioned as the “endothelial zone”. The terms “stromal zone”, “epithelial zone”, and “endothelial zone” mean a compartment essentially comprising, respectively, stromal cells, epithelial cells or endothelial cells, and also their cellular environment, including the molecules that they secrete or synthesize.

According to one particular embodiment, the body of the cell culture substrate or support described above forms the stromal zone described above and the surface of this culture substrate or support forms the base of the epithelial or endothelial zone. In this embodiment, it is possible, for example, to seed stromal cells on the surface of the cell culture support. The stromal cells penetrate the body of the support, migrate and then synthesize the extracellular matrix, allowing progressive filling of the pores of the support. It is also possible to inoculate the stromal cells in an aqueous solution of biopolymer, and then to gel this aqueous solution in order to obtain an aqueous biopolymer gel comprising the seeded cells. Next, the epithelial or endothelial cells are seeded on the surface of the culture support or substrate.

Advantageously, the epithelial zone is distinct from the stromal zone, and comprises a layer of porous material for culturing keratocytes.

Advantageously, the device comprises an insert (or a nacelle) of size suitable for insertion into the recess volume of the device. The cell culture support substrate may be placed in this insert or nacelle. In this embodiment, the aqueous gel of at least one biopolymer is poured into the bottom of the insert. In the case of an aqueous solution, this solution is gelled in the bottom of the insert. The aqueous gel is preferably dehydrated. This gel comprises a substrate or support as defined above.

When an insert is used, the culture medium is preferably introduced into the bottom of the recess and the cells are cultured on the substrate placed in the insert. However, the substrate may also be placed in the bottom of the recess immersed in the culture medium for culturing cells. The aqueous gel is advantageously poured directly into the recess and/or the nacelle. The aqueous gel used to prepare the porous material of the nacelle may be different from that used to prepare the porous material of the recess. Advantageously, the porous material of the nacelle comprises collagen, preferably mixed with a polysaccharide and/or chitosan, optionally modified.

In particular, the insert comprises a shoulder capable of holding the insert in place in the recess by positioning the shoulder on at least part of the edge of the recess.

According to another advantageous feature of the invention, the device is characterized in that it preferably comprises an element for holding at least the peripheral edge of the layer of optionally porous substrate in place, by exerting an anti-shrinkage effect on the substrate, and preferably also ensuring the leak-tightness at the peripheral interface of the said substrate and of the inner side wall, facing it, of the bottom.

In the context of the invention, this peripheral leak-tightness is advantageously provided to prevent exchanges of material at the periphery, i.e. to prevent communication via the lateral edge of the substrate between substances deposited on the top surface of the substrate and the culture medium or the cells that may be present in the substrate.

According to one particular embodiment of the invention, the abovementioned position-maintaining element comprises an annular ring of a size which is sufficient to bear on the peripheral edge of the substrate.

According to another advantageous feature of the invention, the device is characterized in that the said bottom of the nacelle is removable and fastenable to the nacelle, which is itself fastenable to the recess.

According to another advantageous feature of the invention, the device is characterized in that the said removable bottom of the nacelle is fastened by gentle force-fitting or clipping into the recess, thus safely and reliably ensuring the leak-tightness.

According to yet another advantageous feature of the invention, the device is characterized in that the said removable bottom of the nacelle is fastened to the exterior of the recess or of the nacelle, the lower edge of the side wall of the recess or of the nacelle thus constituting an element for holding in place the peripheral edge of the said substrate.

According to another advantageous embodiment of the invention, the device is characterized in that it comprises a plurality of nacelles or inserts per culture well.

According to another advantageous implementation variant of this embodiment, this device also comprises a lid provided with as many orifices as there are nacelles or inserts, each orifice making it possible to receive and to hold in position a nacelle or insert.

According to one advantageous feature of the invention, the device is characterized in that at least the bottom of the recess or of the nacelle or insert containing the porous substrate is sterilized after the said dehydration, and preferably in leaktight packaging.

Advantageously, the device is sterilized. Preferably, the whole culture device is sterilized and advantageously conditioned in leaktight packaging.

According to another advantageous feature of the invention, the abovementioned sterilization is chosen from the group consisting of sterilization by irradiation, preferably with beta rays or gamma rays, or a treatment with a sterilizing gas such as ethylene oxide.

Advantageously, at least some of the inner wall of the bottom or of the nacelle is treated physically, chemically or biologically, or a combination thereof, to promote the cell culture, for example with a coating that promotes the adhesion and/or proliferation of the cells.

In the context of the invention, any physical or chemical process that modifies the overall ionic charge of the material of the wall, advantageously a plastic material, may be used, and/or coating of the wall with any biological molecule that promotes the adhesion and/or proliferation of the cells, such as collagen, fibronectin, laminin, etc. may be used.

According to one variant, at least part of the inner wall of the bottom or of the nacelle may comprise surface reliefs, especially to mimic the striations or cavities of a natural tissue, for instance an epithelium.

According to yet another advantageous embodiment of the invention, the device is characterized in that at least the bottom of the recess or of the nacelle is made of an inert support material chosen, for example, from the group consisting of a synthetic material, a nitrocellulose-based material, a polyamide-based material such as a nylon, a polytetrafluoroethylene- or Teflon®-based material, a polycarbonate-based material, a semi-permeable polyethylene- or polyethylene terephthalate (PET)-based material, a polyester-based material, for example, a cellulose polyester, especially an acetate, a material based on a semi-permeable Biopore-CM® membrane, or polyvinylpyrrolidone.

According to a second aspect, the invention relates to an automated process for preparing a device as defined previously.

Advantageously, a robotizable or automatable platform may be used, preferably to allow positioning of the cell culture devices such as a device for injection or aspiration of culture medium controlled by a robot or an automaton, for example, which is itself controlled by a computer.

Advantageously, the epithelial or endothelial zone is deposited automatically above the stromal zone.

Advantageously, the aqueous gel is poured by an automated device, and/or the cells are seeded by an automated device.

According to a third aspect, the invention relates to leaktight packaging comprising a device as defined previously.

According to a fourth aspect, the invention relates to a model of cornea or of mucous membrane, especially human cornea or mucous membrane, comprising at least corneal or mucous membrane stromal cells (respectively) and at least one support for culturing the corneal or mucous membrane stromal cells, respectively, said support being as defined previously.

Advantageously, the model also comprises corneal or mucous membrane epithelial cells and/or endothelial cells, and preferably corneal or mucous membrane epithelial cells and/or endothelial cells.

The type of seeded cells may have a major impact on the neosynthesis of an extracellular matrix or in the interactions with the epithelium and the endothelium. To reconstruct an artificial stroma similar to corneal stroma, it is advantageous to use keratocytic stem cells or, failing that, keratocytes endowed with high proliferative capacity and still displaying phenotypic characteristics as close as possible to their in vivo counterparts.

The invention also relates to a process for selecting corneal keratocytes or stromal cells as a function of their proliferative capacity, comprising the selection of a predetermined sampling zone on the tissue, preferably human tissue, phenotypic sorting of the sampled corneal keratocytes or stromal cells to select the cells that have a high proliferative capacity.

The invention also relates to the use of a model of cornea, or of mucous membrane, as defined previously as an alternative test model to the toxicity tests especially in cosmetology and pharmacology, on animals.

The invention also relates to the use of a model of cornea defined previously for the preparation of a reconstructed cornea, especially to be used in corneal grafts or in corrective surgery.

Advantageously, the model is prepared in a device as defined previously or via a process as defined previously.

It is understood that, by means of the invention, the various technical problems stated previously are satisfactorily resolved, simply, safely, reliably and reproducibly on the industrial and medical scale, in particular the pharmaceutical and cosmetic scale.

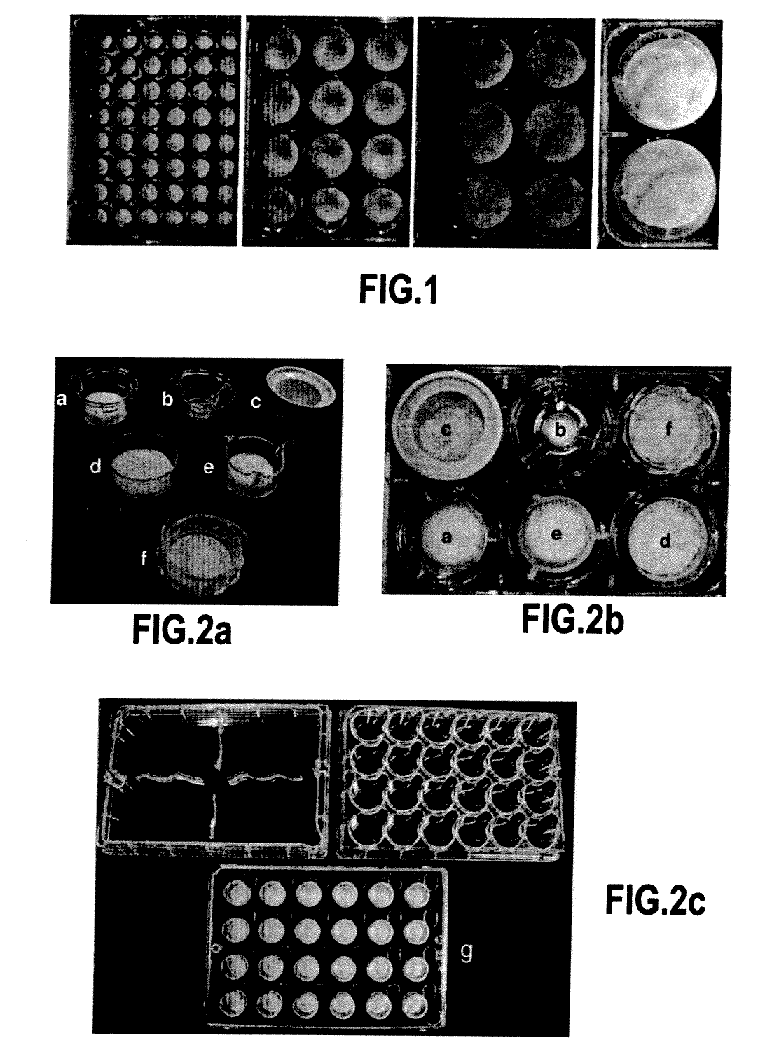

The culture devices described in this invention, and which are advantageously adapted to the use of biomaterial for culturing reconstructed corneas, are presented in FIGS. 1 and 2.

FIG. 1 shows an example of the various culture formats in which the biomaterial according to the present invention is manufactured. From left to right are presented a 96-well plate, a 24-well plate, a 12-well plate and a 6-well plate.

FIG. 2a is a photograph in perspective, and FIG. 2b a photograph in top view, of culture inserts in which the biomaterial according to the invention may be manufactured. a shows a Transwell® insert (Corning, diameter 24 mm), b shows a Snapwell® insert (Corning, diameter 12 mm), c shows a Netwell® insert (Corning, diameter 24 mm), d shows a culture insert (Nunc, diameter 25 mm), e shows a Thincert® insert (Greiner bio-one, diameter 24 mm), f shows a CellCrown insert (Scaffdex, diameter 24 mm). In FIG. 2b, the inserts are positioned in a 6-well culture plate. FIG. 2c shows other culture plates with, at the bottom, a Millicell® plate (Millipore, diameter 9 mm), on the left a collective support plate and on the top right, a 24-well plate.

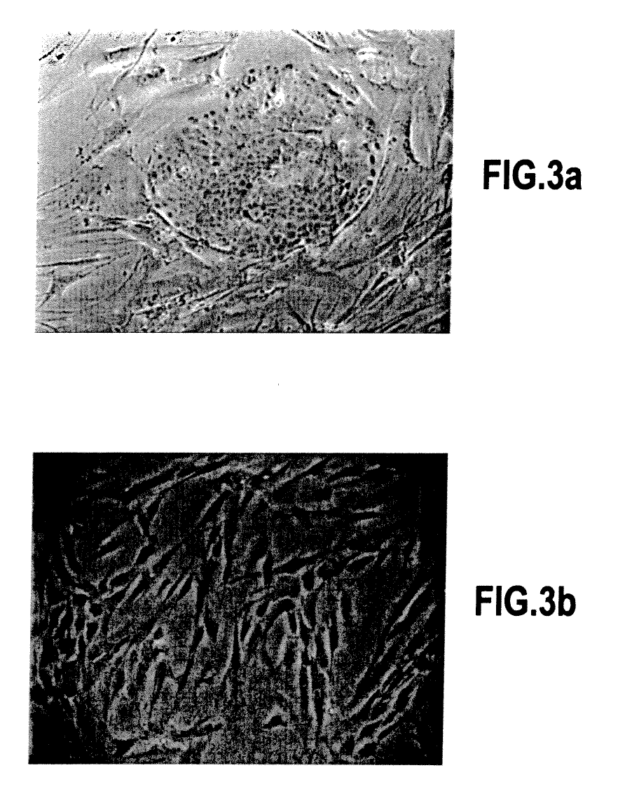

FIG. 3 shows photographs of culturing of epithelial cells and keratocytes of normal human corneas. Photograph 3a shows an in vitro culture of human corneal epithelial cells on a nourishing layer of irradiated fibroblasts. Photograph 3b shows an in vitro culture of human corneal keratocytes.

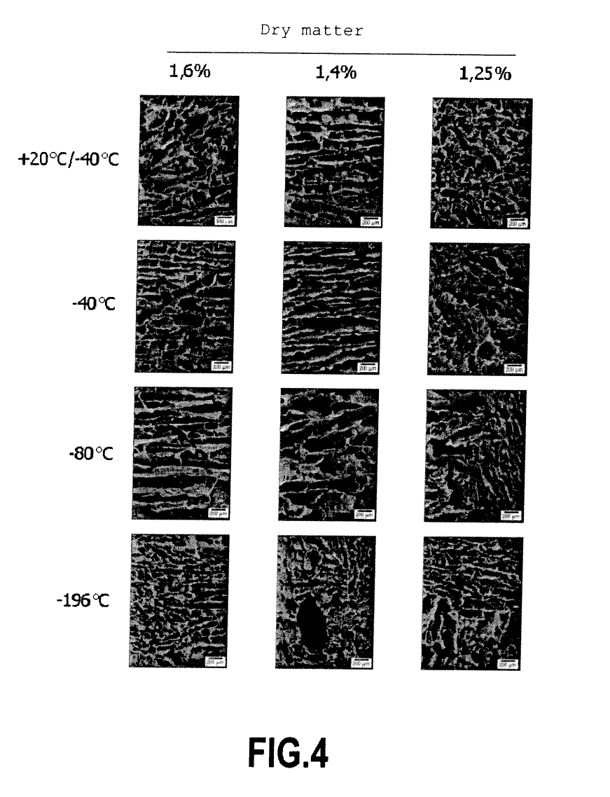

FIG. 4 shows a study of visualization by scanning electron microscopy of a biomaterial according to the present invention, composed of 72% collagen, 8% glycosaminoglycans and 20% chitosan. These visualizations are performed at different percentages of dry matter and at different freeze-drying temperatures.

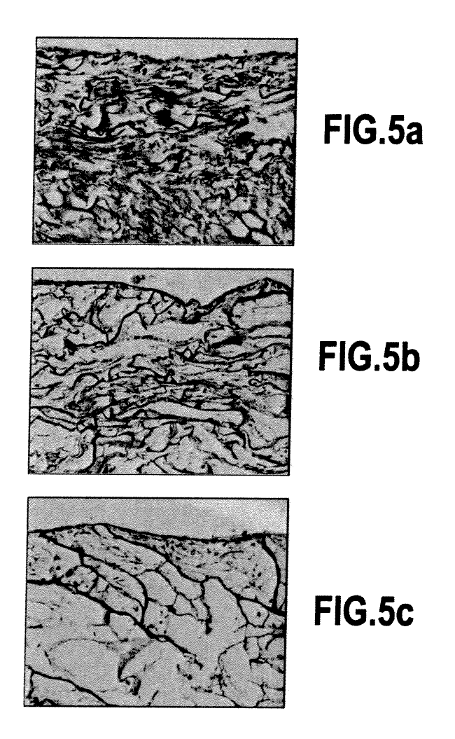

FIG. 5 shows a histological study of the in vitro production of a reconstructed human corneal stroma with a biomaterial of the present invention. FIG. 5a shows a biomaterial comprising 1.6% dry matter and made via a step of freezing at a temperature of −80° C. FIG. 5b shows a biomaterial comprising 1.4% dry matter and made via a step of freezing at a temperature of −40° C. FIG. 5c shows a biomaterial comprising 1.25% dry matter and made via a step of freezing at a temperature of −40° C.

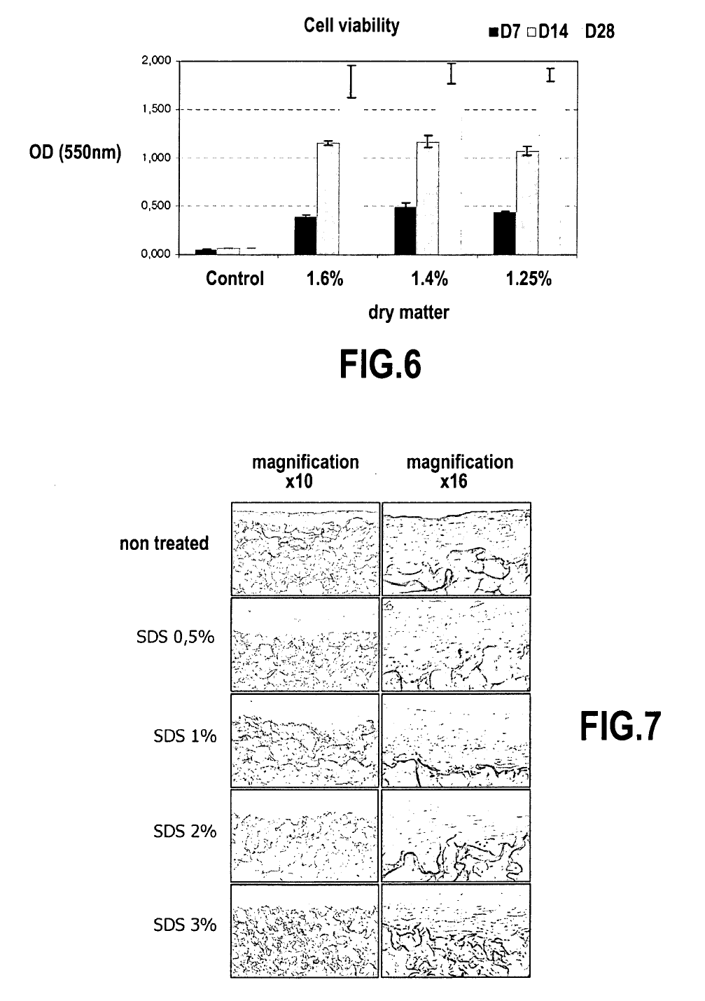

FIG. 6 shows a study of the cell proliferation of keratocytes after in vitro production of a reconstructed human corneal stroma with a biomaterial according to the present invention, at different culturing days (D7, D14, D28), expressed as an OD (optical density) value at 550 nm.

FIG. 7 shows a histological study of the treatment of reconstructed human hemi-corneas with the irritant SDS (sodium dodecyl sulfate).

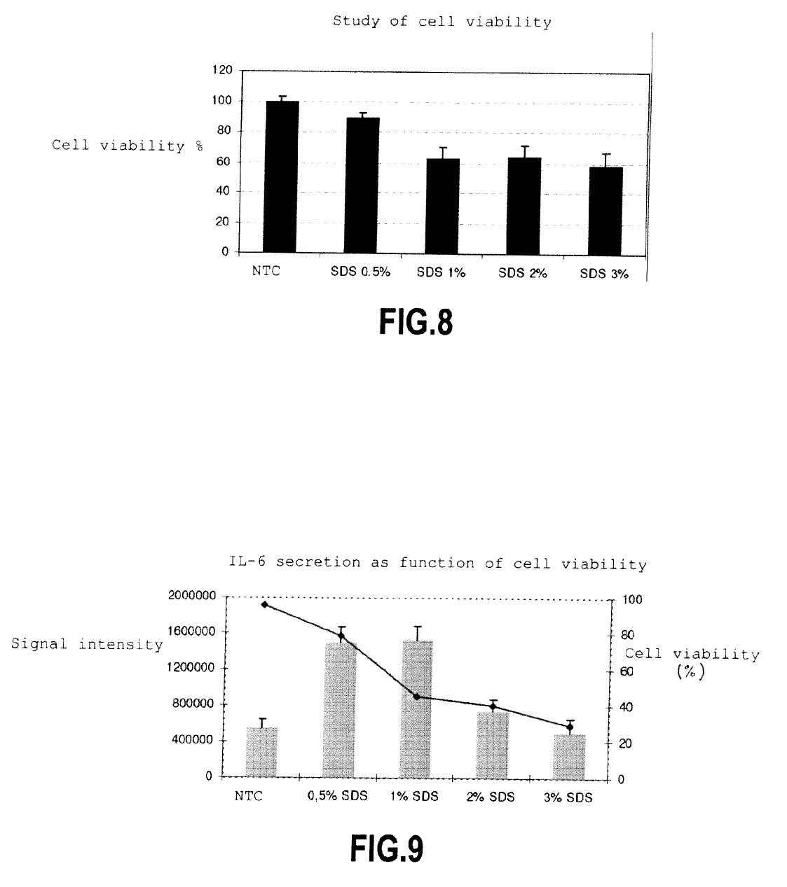

FIG. 8 shows the study of the cell viability after treatment of the reconstructed human hemi-corneas with SDS.

FIG. 9 shows a study of the secretion of a soluble factor (in this case: IL-6 or interleukin-6) by the reconstructed human hemi-corneas treated with SDS.

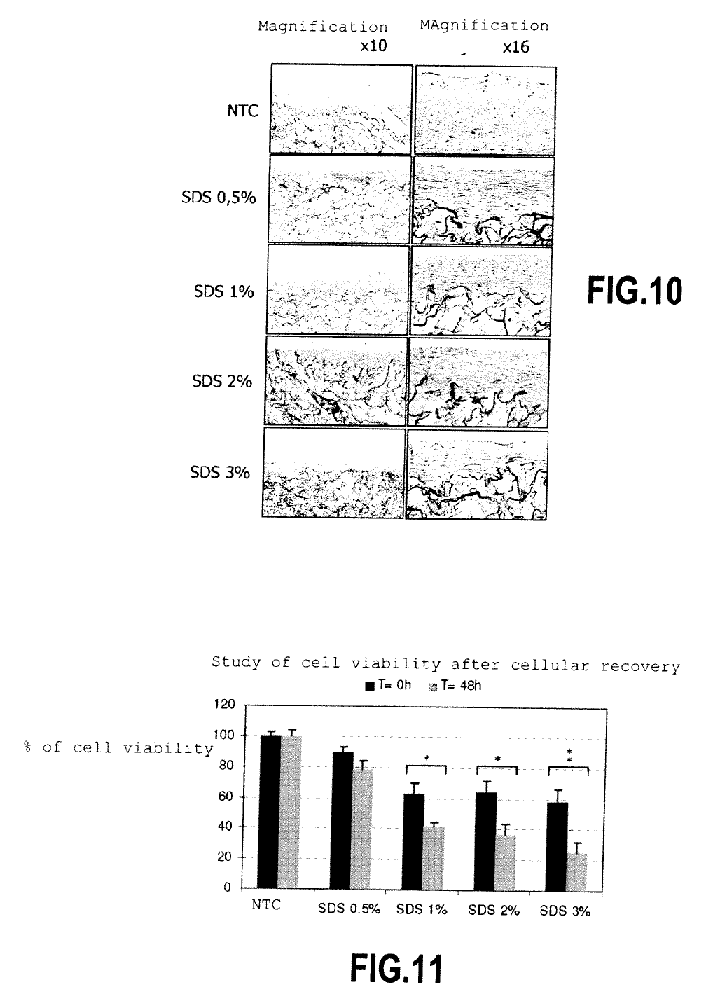

FIG. 10 shows a histological analysis showing the study of the recovery of the reconstructed human hemi-corneas treated with SDS.

FIG. 11 shows an analysis of the cell viability showing the study of the recovery of the reconstructed human hemi-corneas treated with SDS.

Other aims, characteristics and advantages of the invention will emerge clearly to a person skilled in the art on reading the explicative description which makes reference to examples that are given for purely illustrative purposes and that should not in any way be considered as limiting the scope of the invention.

The examples form an integral part of the present invention and any characteristic that appears to be novel relative to any prior art from the description taken as a whole, including the examples, forms an integral part of the invention in its function and in its generality.

Thus, each example has a general scope.

Moreover, in the examples, all the percentages are given on a weight basis, unless otherwise indicated and the temperature is expressed in degrees Celsius unless otherwise indicated, and the pressure is atmospheric pressure, unless otherwise indicated.

### EXAMPLES

### Example 1

### Culturing of Normal Human Corneal Epithelial Cells and Keratocytes (FIG. 3)

The human corneas are collected according to the rules of ethics and cannot be used therapeutically due to an excessively low endothelial density. The corneas are stored as an organoculture at 31° C. until the time of extraction of the epithelial cells and keratocytes.

Extraction and Culturing of Human Corneal Epithelial Cells

The corneas are incubated in the presence of trypsin/EDTA (0.05%/0.01%) for 80 minutes at 37° C. in order to recover the epithelial compartment from the biopsy. The epithelium is then dissociated in the presence of trypsin/EDTA (0.05%/0.01%) and the epithelial cells obtained are seeded, preferentially at a density of 1.5×10⁴ cells/cm², on a culture support such as a nourishing layer of irradiated fibroblasts and in a culture medium advantageously composed of DMEM/HAM F12 (2/1), 10% foetal calf serum (FCS), insulin (5 μg/ml), adenine (0.18 mM), hydrocortisone (0.4 μg/ml), cholera toxin (0.1 nM), triiodothyronine (2 nM), glutamine (4 mM), penicillin G (100 IU/ml), gentamicin (20 μg/ml) and amphotericin B (1 μg/ml). EGF (10 ng/ml) is preferentially added after 3 days of culturing. The culturing is performed at 37° C. and 5% CO₂ under a humid atmosphere.

The epithelial cells cultured in vitro (FIG. 3a) may be frozen and/or reseeded in culture, for example on a nourishing layer of irradiated fibroblasts and/or used for the in vitro production of reconstructed human hemi-corneas/corneas.

Extraction and Culturing of Human Corneal Keratocytes

The corneas are incubated in the presence of trypsin/EDTA (0.05%/0.01%) for 80 minutes at 37° C. in order to recover the stromal compartment from the biopsy. The stroma is then incubated for 3 hours at 31° C. and with stirring in the presence of collegenase A (3 mg/ml). The cell extract obtained is then purified, for example on a 70 μm screen, and then cultured, preferentially at a density of 1.0×10⁴ cells/cm², in a culture medium advantageously composed of DMEM/HAM F12, 10% neonatal calf serum, b-FGF (5 ng/ml), penicillin G (100 IU/ml), gentamicin (20 μg/ml) and amphotericin B (1 μg/ml).

The keratocytes cultured in vitro (FIG. 3b) may be frozen and/or reseeded in culture and/or used for the in vitro production of reconstructed human corneal stroma and hemi-corneas/corneas.

### Example 2

### Study of the Porosity of the Biomaterial According to the Invention Based on Collagen, Glycosaminoglycan and Chitosan for the In Vitro Production of Reconstructed Human Corneal Stroma and Hemi-Corneas/Corneas (FIG. 4)

In order to demonstrate that the size of the pores of the biomaterial is a fundamental parameter in the in vitro production of reconstructed human corneal stroma and hemi-corneas/corneas, the inventors varied its porosity from 30 μm to 100 μm. To do this, the inventors preferentially modified two manufacturing parameters that have a direct influence on the pore diameter of the biomaterial: (1) the percentage of dry matter in the aqueous composition of collagen (72%), glycos-aminoglycan (GAG) (8%) and chitosan (20%), (2) the kinetics of the freezing temperatures of the aqueous gel before its dehydration by freeze-drying.

Three compositions of aqueous gel, whose collagen/GAG/chitosan ratio does not change (72%/8%/20%, respectively), were prepared in the following manner:
an aqueous gel containing 2% dry matter, i.e. 5.6 g of collagen +1.56 g of chitosan +0.62 g of GAG was prepared;
an aqueous gel containing 1.6% dry matter, prepared by dilution with water of the 2% gel;
an aqueous gel containing 1.4% dry matter, prepared by dilution with water of the 2% gel;
an aqueous gel containing 1.25% dry matter, prepared by dilution with water of the 2% gel.

The aqueous gels were then poured in culture inserts of Snapwell® type at an amount of 450 mg/insert.

Freeze-drying of the aqueous gels (in the Snapwell® inserts) is then performed at −40° C. This freeze-drying step requires a prior freezing step, which was performed according to four variants and in the following manner:
direct freezing of the aqueous gels at −40° C.,
direct freezing of the aqueous gels at −80° C.,
direct freezing of the aqueous gels at −196° C.,
gradual freezing of the aqueous gels from +20° C. to −40° C. (freezing time preferably of 30 to 45 minutes minimum).

The evaluation of the porosity of the biomaterial is performed by scanning electron microscopy studies at magnifications ranging from 50× to 7000×, and by automatic image analysis studies. The results are given in FIG. 4. The manufacturing conditions selected for preparing a biomaterial having a porosity of between 40 and 50 μm are as follows:
an aqueous gel containing 1.6% dry matter followed by a step of freezing at −80° C. has a porosity of 42.3 μm,
an aqueous gel containing 1.4% dry matter followed by a step of freezing at −40° C. has a porosity of 48.5 μm,
an aqueous gel containing 1.25% dry matter followed by a step of freezing at −40° C. has a porosity of 47.3 μm.

### Example 3

### In Vitro Production of Reconstructed Human Corneal Stroma with Our Porous Biomaterial: Histological Study (FIG. 5)

To demonstrate that the biomaterial that has a porosity of between 40 and 50 μm is suitable for culturing human keratocytes, the inventors studied the capacity of the keratocytes to become integrated and organized in the biomaterial, in the course of in vitro culturing of a reconstructed human corneal stroma.

The histological study of the reconstructed human corneal stroma in the porous biomaterial was performed in the following manner:

Preparation of the keratocytes: as described in Example 1.

Preparation of the porous biomaterial: as described in Example 2.

Preparation of the Reconstructed Human Corneal stroma:

A cell suspension preferentially containing 2×10⁵ keratocytes/500 μl of culture medium is seeded on the surface of our porous biomaterial. The culture medium used is preferentially composed of DMEM/HAM F12, 10% neonatal calf serum, b-FGF (5 ng/ml), ascorbic acid (1 mM), penicillin G (100 IU/ml), gentamicin (20 μg/ml) and amphotericin B (1 μg/ml). The keratocytes are then cultured, for example, for 14 days in the culture medium described above.

Histological Study:

After culturing for 14 days, the reconstructed human corneal stromas are rinsed with culture medium, for example, DMEM, and then fixed, for example, with formaldehyde, and then included in paraffin. Paraffin slices are then prepared by microtome, preferentially at a thickness of 5 μm, and then stained, for example, with haematoxylin, phloxin and saffron. The results are given in FIG. 5: a/aqueous gel containing 1.6% solid matter followed by a step of freezing at −80° C. (porosity of 42.3 μm), b/aqueous gel containing 1.4% solid matter followed by a step of freezing at −40° C. (porosity of 48.5 μm), c/aqueous gel containing 1.25% solid matter followed by a step of freezing at −40° C. (porosity of 47.3 μm). The keratocytes cultured in the porous biomaterial (porosity of between 40 and 50 μm) are capable of organizing into tissue with the formation of several cell layers and the synthesis of their own extracellular matrix.

### Example 4

### In Vitro Production of Reconstructed Human Corneal Stromas with the Porous Biomaterial: Study of the Cellular Proliferation of Keratocytes (FIG. 6)

Following Example 3, the inventors then studied the proliferative capacity of keratocytes in the course of in vitro culturing of a reconstructed human corneal stroma.

The study of the cellular proliferation of the keratocytes in the porous biomaterial was performed in the following manner:

Preparation of the Keratocytes: as Described in Example 1.

Preparation of the porous biomaterial: as described in Example 2.

Preparation of the reconstructed human corneal stroma: as described in Example 3.

Study of the Cellular Proliferation of the Keratocytes in a Reconstructed Human Corneal Stroma:

After culturing for 7, 14 or 28 days, the reconstructed human corneal stromas are analysed in the following manner:
the cultures are incubated for 2 hours at 37° C. in 2 ml of MTT (methyl thiazolyl tetrazolium) at 1 mg/ml. The negative control corresponds to the biomaterial not containing keratocytes;
the cultures are then recovered, and then incubated in 4 ml of DMSO (dimethyl sulfoxide) for 4 hours with stirring;
a fraction of the culture supernatant (200 μl) of each sample is recovered and then analysed by spectrophotocolorimetry (550 nm). The optical density read is directly proportional to the number of living cells. The results are presented in FIG. 6. The biomaterial that has a porosity of between 40 and 50 μm is suitable for the proliferation of keratocytes. Indeed, the cellular proliferation kinetics of the keratocytes is proportional to the culturing time of the reconstructed human corneal stromas. Furthermore, irrespective of the conditions of manufacture of our biomaterial [1.6%, 1.4% and 1.25% dry matter], as described in Example 2, the keratocytes have a similar degree of proliferation.

### Example 5

### In Vitro Production of a Reconstructed Human Hemi-Cornea as a Pharmacotoxicological Model: Histological Study (FIG. 7)

The inventors studied the effect of the irritant SDS (sodium dodecyl sulfate) on human hemi-corneas reconstructed with the biomaterial.

The histological study of the reconstructed human hemi-corneas, after treatment with SDS, is performed in the following manner:

Preparation of the cells: as described in Example 1.

Preparation of the porous biomaterial: as described in Example 2. For this study, the inventors used the aqueous gel [collagen (72%), GAG (8%) and chitosan (20%)] containing 1.6% dry matter, which was then subjected to the step of freezing at −80° C.

Preparation of the reconstructed human corneal stroma: as described in Example 3.

Preparation of the reconstructed human hemi-corneas:

A cell suspension preferentially containing 2×10⁵ corneal epithelial cells/500 μl of culture medium is seeded on the surface of the reconstructed human corneal stroma. The culture medium used is preferentially composed of DMEM/HAM F12 (2/1), 10% foetal calf serum (FCS), EGF (10 ng/ml), insulin (5 μg/ml), adenine (0.18 mM), hydrocortisone (0.4 μg/ml), cholera toxin (0.1 nM), triiodothyronine (2 nM), glutamine (4 mM), ascorbic acid (1 mM), penicillin G (100 IU/ml), gentamicin (20 μg/ml) and amphotericin B (1 μg/ml). The corneal epithelial cells are cultured by immersion for 7 days on the equivalent corneal stroma and with the medium described above.

The cultures are then raised to the air/liquid interface and then cultured for a further 14 days with a culture medium preferentially composed of DMEM, hydrocortisone (0.4 μg/ml), insulin (5 μg/ml), ascorbic acid (1 mM), penicillin G (100 IU/ml), gentamicin (20 μg/ml) and amphotericin B (1 μg/ml).

Treatment of Reconstructed Human Hemi-Corneas With SDS:

The reconstructed human hemi-corneas are treated for 10 minutes by complete immersion, with increasing solutions of SDS (0.5%, 1%, 2% and 3%). After the treatment, the reconstructed human hemi-corneas are rinsed with culture medium, for example DMEM, and then fixed, for example with formaldehyde, and then included in paraffin. Paraffin slices are then prepared by microtome, preferentially at a thickness of 5 μm, and then stained, for example, with haematoxylin, phloxin and saffron. The histological results are given in FIG. 7. The inventors showed that there is a direct correlation between the increasing SDS concentrations and the disorganization of the superficial cellular layers of the reconstructed corneal epithelium. At and above 0.5% SDS, a histological effect on the reconstructed human hemi-corneas is observed.

### Example 6

### In Vitro Production of a Reconstructed Human Hemi-Cornea as a Pharmacotoxicological Model: Study of the Cell Viability (FIG. 8):

Following Example 5, the inventors also studied the cell viability of reconstructed human hemi-corneas treated with the irritant SDS, in the following manner:

Preparation of the cells: as described in Example 1.

Preparation of the porous biomaterial: as described in Example 2. For this study, the inventors used the aqueous gel [collagen (72%), GAG (8%) and chitosan (20%)] containing 1.6% solid matter, which was then subjected to the step of freezing at −80° C.

Preparation of the reconstructed human corneal stroma: as described in Example 3.

Preparation of the reconstructed human hemi-corneas treated with SDS: as described in Example 5.

Study of the cell viability of the reconstructed human hemi-corneas treated with SDS: as described in Example 4 for a reconstructed human corneal stroma.

The cell viability results are given in FIG. 8. The inventors showed that there is a direct correlation between the increasing SDS concentrations and the percentage of cell viability. Beyond 0.5% SDS, the cell viability of the reconstructed human hemi-corneas is no longer satisfactory and is less than 75%. The biological effects of SDS on the reconstructed human hemi-corneas should thus be evaluated at concentrations close to 0.5%.

### Example 7

### In Vitro Production of a Reconstructed Human Hemi-Cornea as a Pharmacotoxicological Model: Study of the Secretion of Soluble Factors (FIG. 9)

Following Examples 5 and 6, the inventors studied the secretion of soluble factors, for instance pro-inflammatory cytokines, produced by the reconstructed human hemi-corneas treated with the irritant SDS. This study was performed in the following manner:

Preparation of the cells: as described in Example 1.

Preparation of the porous biomaterial: as described in Example 2. For this study, the inventors used the aqueous gel [collagen (72%), GAG (8%) and chitosan (20%)] containing 1.6% dry matter, which was then subjected to the step of freezing at −80° C.

Preparation of the reconstructed human corneal stroma: as described in Example 3.

Preparation of the reconstructed human hemi-corneas treated with SDS: as described in Example 5.

Study of the Secretion of Pro-Inflammatory Cytokines by the Reconstructed Human Hemi-Corneas Treated with SDS:

After treating with SDS, the reconstructed human hemi-corneas are rinsed with culture medium, for example, DMEM, and then recultured for at least a further 24 hours in the culture medium described for the preparation of the reconstructed human hemi-corneas (Example 5).

The culture supernatants of the reconstructed human hemi-corneas are then recovered in order to assay the secreted pro-inflammatory cytokines. The culture supernatants optionally may be frozen before use.

The inventors evaluated the secretion of IL-6, for example by means of a “membrane ARRAY” method as a function of the cell viability previously studied in Example 6. The results of the IL-6 assay are given in FIG. 9. The inventors showed that there is a direct correlation between a strong secretion of IL-6 and the irritant effect of SDS without cell mortality.

### Example 8

### In Vitro Production of a Reconstructed Human Hemi-Cornea as a Pharmacotoxicological Model: Study of the Cellular Recovery by Histological Analysis (FIG. 10)

Following Examples 5 to 7, the inventors studied the cellular recovery of reconstructed human hemi-corneas treated with SDS, i.e. their capacity for regeneration following the effect of a treatment, for example, in this study, following the effect of an irritant. This recovery study was performed in the following manner:

Preparation of the cells: as described in Example 1.

Preparation of the porous biomaterial: as described in Example 2. For this study, the inventors used the aqueous gel [collagen (72%), GAG (8%) and chitosan (20%)] containing 1.6% dry matter, which was then subjected to the step of freezing at −80° C.

Preparation of the reconstructed human corneal stroma: as described in Example 3.

Preparation of the reconstructed human hemi-corneas treated with SDS: as described in Example 5.

Study of the Cellular Recovery of the Reconstructed Human Hemi-Corneas Treated with SDS:

After treatment with SDS, the reconstructed human hemi-corneas are rinsed with culture medium, for example, DMEM, and then recultured for at least a further 24 hours in the culture medium described for the preparation of the reconstructed human hemi-corneas (Example 5). The histological studies are performed as described in Example 5. The histological results are given in FIG. 10. The inventors showed that the reconstructed human hemi-corneas treated with 0.5% and 1% SDS have, after 48 hours of cellular recovery, an epithelial organization close to that of untreated hemi-corneas. On the other hand, the treatments with 2% and 3% SDS induce irreversible epithelial damage.

### Example 9

### In Vitro Production of a Reconstructed Human Hemi-Cornea as a Pharmacotoxicological Model: Study of the Cellular Recovery by Analysis of the Cell Viability (FIG. 11)

Following Example 8, the inventors then studied the cell viability of reconstructed human hemi-corneas after at least 24 hours of recovery, following treatment with the irritant SDS. This recovery study was performed in the following manner:

Preparation of the cells: as described in Example 1.

Preparation of the porous biomaterial: as described in Example 2. For this study, the inventors used the aqueous gel [collagen (72%), GAG (8%) and chitosan (20%)] containing 1.6% dry matter, which was then subjected to the step of freezing at −80° C.

Preparation of the reconstructed human corneal stroma: as described in Example 3.

Preparation of the reconstructed human hemi-corneas treated with SDS: as described in Example 5.

Study of the cellular recovery of the reconstructed human hemi-corneas treated with SDS: as described in Example 8.

The cell viability studies are performed as described in Example 6. The results are given in FIG. 11. The inventors showed that the reconstructed human hemi-corneas treated with 0.5% SDS and after recovery for at least 24 hours (T=48 h) have a cell viability that is significantly equivalent to that of the reconstructed human hemi-corneas after treatment (T=0 h). On the other hand, the reconstructed human hemi-corneas treated with 1%, 2% and 3% SDS have a significantly lower cell viability after at least 24 hours of recovery (T=48 h) than after the treatment (T=0 h).

1-18. (canceled)

19. Cell culture support for culturing corneal cells, said support comprising at least one biopolymer in the form of a porous matrix whose porosity is comprised between 10 and 100 microns.

20. Support according to claim 19 characterized in that porosity is comprised between 30 and 70 microns and more preferably between 40 and 50 microns.

21. Support according to claim 19, characterized in that the porous matrix is based on an aqueous solution of at least one biopolymer, preferably of a macromolecule of the extracellular matrix (ECM), preferably a glycoprotein and more preferably based on an aqueous collagen solution.

22. Support according to claim 19, characterized in that the porous matrix comprises at least two biopolymers as a mixture.

23. Support according to claim 19, characterized in that the porous matrix comprises at least one glycoprotein, and preferably collagen, optionally in combination with one or more optionally substituted polysaccharides, for instance a glycosaminoglycan, such as chondroitin 4-sulfate, chondroitin 6-sulfate or hyaluronic acid, or a mixture thereof, and/or chitin, and/or chitosan; and/or one or more proteoglycans; and preferably with at least one polysaccharide and chitosan, optionally modified.

24. Support according to claim 19, characterized in that the porous matrix is obtained from a dehydrated aqueous gel.

25. Support according to claim 19, characterized in that the porous matrix is based on a mixture of collagen, of at least one polysaccharide, and of chitosan, optionally modified, and preferably with a proportion of collagen of between 60% and 90%, of GAG of between 0 and 15%, and of chitosan of between 10% and 30%, as a dry weight percentage relative to the total dry weight of this mixture.

26. Support according to claim 19, characterized in that the aqueous gel based on a mixture of collagen, glycosaminoglycan and chitosan, optionally modified, has a concentration of between 1.25% and 1.6% of the mixture of collagen, glycosaminoglycan and chitosan, optionally modified, relative to the aqueous medium (w/w).

27. Support according to claim 19, characterized in that the porous matrix is obtained by freeze-drying an aqueous gel, especially by freezing at a temperature of between −30° C. and −196° C., and preferably, for industrial reasons, at a temperature of between −40° C. and −80° C.

28. Cell culture device for corneal cells, characterized in that the cell culture device comprises a recess and at least one support as defined in claim 19, forming a layer for culturing the cells to be cultured, this layer being obtained by dehydration of an aqueous gel of at least one biopolymer poured directly into the recess of the device.

29. Device according to claim 28, characterized in that it comprises a first zone for culturing stromal cells, known as the “stromal zone”, and a second zone for culturing either epithelial cells, known as the “epithelial zone”, or endothelial cells, known as the “endothelial zone”.

30. Device according to claim 28, characterized in that it comprises a first zone for culturing stromal cells, known as the “stromal zone”, and a second zone for culturing epithelial cells, known as the “epithelial zone”, and a third zone for culturing endothelial cells, known as the “endothelial zone”.

31. Device according to claim 28, characterized in that the stromal zone comprises the culture support placed in an insert (or nacelle) of dimension suitable for insertion into the volume of the recess of the device.

32. Device according to claim 28, characterized in that the epithelial zone is distinct from the stromal zone, and comprises a layer of porous material for culturing keratocytes.

33. Model of corneal stroma, especially human corneal stroma, comprising at least corneal stromal cells, preferably corneal keratocytes and at least one support for culturing the corneal stromal cells, the said support being as defined in claim 19.

34. Model of cornea, especially human cornea, comprising a model of corneal stroma according to claim 33 and corneal epithelial cells and/or endothelial cells, and preferably corneal epithelial cells and endothelial cells.

35. Use of a model as defined in claim 33, as an alternative test model to the toxicity tests on animals, especially in cosmetology and pharmacology.

36. Use of a model of corneal stroma as defined in claim 33 or of a model of cornea as defined in claim 34, for the preparation of a reconstructed cornea, especially for use in a corneal graft or in corrective surgery.
